(19) United States
(12) Patent Application Publication
(10) Pub. No.: US 2019/0015160 A1
(43) Pub. Date: Jan. 17, 2019

(54) MEDICAL IMAGE DIAGNOSIS APPARATUS, MEDICAL INFORMATION PROCESSING APPARATUS, AND MEDICAL INFORMATION PROCESSING METHOD

(71) Applicant: Canon Medical Systems Corporation, Otawara-shi (JP)

(72) Inventor: Tatsuo MAEDA, Nasushiobara (JP)

(73) Assignee: Canon Medical Systems Corporation, Otawara-shi (JP)

(21) Appl. No.: 16/033,633

(22) Filed: Jul. 12, 2018

(30) Foreign Application Priority Data
Jul. 14, 2017 (JP) 2017-138061

(51) Int. Cl. A61B 34/10; A61B 6/03; A61B 6/00; A61B 8/14

(52) U.S. Cl. CPC A61B 34/10; A61B 6/032; A61B 2034/2063; A61B 8/14; A61B 2034/107; A61B 6/5247

(57) ABSTRACT

A medical image diagnosis apparatus according to an embodiment includes processing circuitry. The processing circuitry is configured to obtain volume data acquired by imaging a subject by using a first image taking apparatus. The processing circuitry is configured to set one or more insertion paths to be used when a device is inserted into the subject, on a basis of the volume data. The processing circuitry is configured to obtain an imaged range that is related to the subject and is to be used by a second image taking apparatus when the device is inserted into the subject. The processing circuitry is configured to evaluate the one or more insertion paths by comparing the set insertion paths with the imaged range. The processing circuitry is configured to present a result of the evaluation.

### CROSS-REFERENCE TO RELATED APPLICATIONS

This application is based upon and claims the benefit of priority from Japanese Patent Application No. 2017-138061, filed on Jul. 14, 2017; the entire contents of which are incorporated herein by reference.

### FIELD

Embodiments described herein relate generally to a medical image diagnosis apparatus, a medical information processing apparatus, and a medical information processing method.

### BACKGROUND

Conventionally, to carry out puncture treatment, a puncture plan is made to determine a site subject to a puncture procedure (a target), a puncture insertion point (an entry point), and an insertion path for the puncture (a puncture route), while using a medical image (e.g., an X-ray image, an X-ray CT image, or the like) for a puncture planning purpose that was acquired in advance. The puncture treatment is then carried out on the basis of the puncture plan that was made. For example, to make such a puncture plan, the puncture insertion point is set in such a manner that blood vessels, bones, and the like are not included in the puncture route. The setting process is performed either manually or automatically.

### BRIEF DESCRIPTION OF THE DRAWINGS

FIG. 1 is a diagram illustrating an exemplary configuration of an X-ray diagnosis apparatus according to a first embodiment;

FIG. 2 is a drawing illustrating an example of a target setting process according to the first embodiment;

FIG. 3 is a drawing illustrating an example of a process of extracting puncture route candidate regions performed by a controlling function according to the first embodiment;

FIG. 4 is a drawing for explaining movability of a C-arm according to the first embodiment;

FIG. 5 is a drawing illustrating an example of an interference range obtained by an obtaining function according to the first embodiment;

FIG. 6 is a drawing for explaining an example of information about interference obtained by the obtaining function according to the first embodiment;

FIG. 7 is a drawing illustrating examples of regions extracted by an extracting function according to the first embodiment;

FIG. 8 is a drawing illustrating an example of a display presented on a display by a presenting function according to the first embodiment;

FIG. 9 is a drawing illustrating another example of a display presented on the display by the presenting function according to the first embodiment;

FIG. 10 is a flowchart illustrating a processing procedure performed by an X-ray diagnosis apparatus according to the first embodiment;

FIG. 11 is a drawing illustrating an example of an attachment according to a second embodiment;

FIG. 12 is a drawing for explaining an example of a tilting angle of a gantry included in an X-ray CT apparatus according to the second embodiment;

FIG. 13 is a drawing illustrating an example of an ultrasound probe according to the second embodiment; and

FIG. 14 is a diagram illustrating an exemplary configuration of a medical information processing apparatus according to the second embodiment.

### DETAILED DESCRIPTION

According to an embodiment, a medical image diagnosis apparatus includes processing circuitry. The processing circuitry is configured to obtain volume data acquired by imaging a subject by using a first image taking apparatus. The processing circuitry is configured to set one or more insertion paths to be used when a device is inserted into the subject, on a basis of the volume data. The processing circuitry is configured to obtain an imaged range that is related to the subject and is to be used by a second image taking apparatus when the device is inserted into the subject. The processing circuitry is configured to evaluate the one or more insertion paths by comparing the set insertion paths with the imaged range. The processing circuitry is configured to present a result of the evaluation.

Exemplary embodiments of a medical image diagnosis apparatus, a medical information processing apparatus, and a medical information processing method will be explained in detail below, with reference to the accompanying drawings. In a first embodiment, an example will be explained in which an X-ray diagnosis apparatus is used as a medical image diagnosis apparatus of the present disclosure. Further, possible embodiments of the medical image diagnosis apparatus, the medical information processing apparatus, and the medical information processing method of the present disclosure are not limited to the embodiments described below.

### First Embodiment

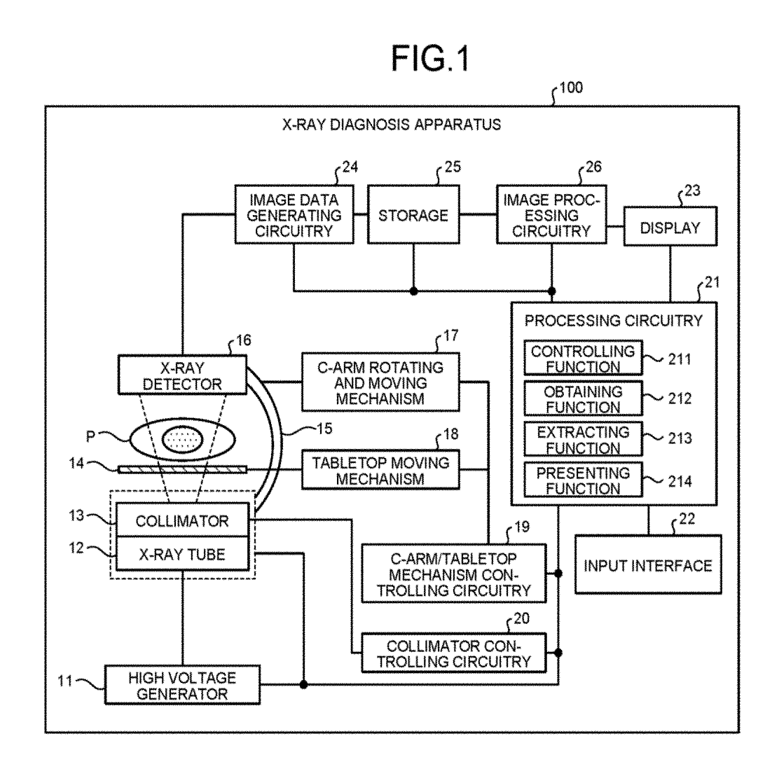

First, an overall configuration of the X-ray diagnosis apparatus according to the first embodiment will be explained. FIG. 1 is a diagram illustrating an exemplary configuration of an X-ray diagnosis apparatus 100 according to the first embodiment. As illustrated in FIG. 1, the X-ray diagnosis apparatus 100 according to the first embodiment includes a high voltage generator 11, an X-ray tube (a tube bulb) 12, a collimator 13, a tabletop 14, a C-arm (a supporting member) 15, an X-ray detector 16, a C-arm rotating and moving mechanism 17, a tabletop moving mechanism 18, C-arm/tabletop mechanism controlling circuitry 19, collimator controlling circuitry 20, processing circuitry 21, an input interface 22, a display 23, image data generating circuitry 24, storage 25, and image processing circuitry 26.

In the X-ray diagnosis apparatus 100 illustrated in FIG. 1, processing functions thereof are stored in the storage 25 in the form of computer-executable programs. The C-arm/tabletop mechanism controlling circuitry 19, the collimator controlling circuitry 20, the processing circuitry 21, the image data generating circuitry 24, and the image processing circuitry 26 are processors configured to realize the functions corresponding to the programs by reading and executing the programs from the storage 25. In other words, each of the circuits that has read the corresponding one of the programs has the function corresponding to the read program.

The high voltage generator 11 is configured, under control of the processing circuitry 21, to generate high voltage and to supply the generated high voltage to the X-ray tube 12. The X-ray tube 12 is configured to generate X-rays by using the high voltage supplied thereto from the high voltage generator 11.

The collimator 13 is configured to limit the X-rays generated by the X-ray tube 12 so as to be selectively radiated onto a region of interest of an examined subject P, under control of the collimator controlling circuitry 20. For example, the collimator 13 includes four slidable collimator blades. Under the control of the collimator controlling circuitry 20, the collimator 13 limits the X-rays generated by the X-ray tube 12 by sliding the collimator blades and causes the limited X-rays to be radiated onto the subject P. Further, the collimator 13 includes an additional filter for adjusting the quality of the X-rays. The additional filter may be configured in accordance with the medical examination to be performed, for example. The tabletop 14 is a bed on which the subject P is placed and is arranged over a couch (not illustrated). The subject P is not included in the X-ray diagnosis apparatus 100.

The X-ray detector 16 is configured to detect X-rays that have passed through the subject P. For example, the X-ray detector 16 includes detecting elements arranged in a matrix formation. The detecting elements are configured to convert the X-rays that have passed through the subject P into electrical signals, to accumulate the electrical signals therein, and to transmit the accumulated electrical signals to the image data generating circuitry 24.

The C-arm 15 is configured to hold the X-ray tube 12, the collimator 13, and the X-ray detector 16. The C-arm 15 is caused to rotate at a high speed like a propeller around the subject P lying on the tabletop 14, by a motor provided in a supporting unit (not illustrated). In this situation, the C-arm 15 is supported so as to be rotatable on three axes, namely X-, Y-, and Z-axes, that are orthogonal to one another. By a driving unit (not illustrated), the C-arm can be caused to rotate on each of the axes individually. The X-ray tube 12 with the collimator 13 and the X-ray detector 16 are arranged by the C-arm 15 so as to oppose each other while the subject P is interposed therebetween. Although FIG. 1 illustrates the example in which the X-ray diagnosis apparatus 100 is a single-plane apparatus, possible embodiments are not limited to this example. The X-ray diagnosis apparatus 100 may be a bi-plane apparatus.

The C-arm rotating and moving mechanism 17 is a mechanism used for rotating and moving the C-arm 15. Further, the C-arm rotating and moving mechanism 17 is also capable of changing the Source Image receptor Distance (SID), which is the distance between the X-ray tube 12 and the X-ray detector 16. Further, the C-arm rotating and moving mechanism 17 is also capable of rotating the X-ray detector 16 held by the C-arm 15. The tabletop moving mechanism 18 is a mechanism used for moving the tabletop 14.

The C-arm/tabletop mechanism controlling circuitry 19 is configured, under the control of the processing circuitry 21, to adjust the rotating and the moving of the C-arm 15 and the moving of the tabletop 14, by controlling the C-arm rotating and moving mechanism 17 and the tabletop moving mechanism 18. For example, under the control of the processing circuitry 21, the C-arm/tabletop mechanism controlling circuitry 19 controls a rotating imaging process in which projection data is acquired at a predetermined framerate while causing the C-arm 15 to rotate. The collimator controlling circuitry 20 is configured, under the control of the processing circuitry 21, to control the radiation range of the X-rays radiated onto the subject P, by adjusting the opening degree of the collimator blades included in the collimator 13.

The image data generating circuitry 24 is configured to generate projection data by using the electrical signals converted from the X-rays by the X-ray detector 16 and to store the generated projection data into the storage 25. For example, the image data generating circuitry 24 generates the projection data by applying a current/voltage conversion, an Analog/Digital (A/D) conversion, and/or a parallel/serial conversion to the electrical signals received from the X-ray detector 16. Further, the image data generating circuitry 24 stores the generated projection data into the storage 25.

The storage 25 is configured to receive and store therein the projection data generated by the image data generating circuitry 24. Further, the storage 25 has stored therein the programs corresponding to the various types of functions that are read and executed by the circuits illustrated in FIG. 1. In one example, the storage 25 has stored therein a program corresponding to a controlling function 211, a program corresponding to an obtaining function 212, a program corresponding to an extracting function 213, and a program corresponding to a presenting function 214 that are read and executed by the processing circuitry 21.

The image processing circuitry 26 is configured, under the control of the processing circuitry 21 (explained later), to generate an X-ray image by performing any of various types of image processing processes on the projection data stored in the storage 25. As another example, the image processing circuitry 26 is configured, under the control of the processing circuitry 21 (explained later), to generate an X-ray image by obtaining projection data directly from the image data generating circuitry 24 and performing any of various types of image processing processes on the obtained projection data. The image processing circuitry 26 is also capable of storing the X-ray images resulting from the image processing processes into the storage 25. For example, the image processing circuitry 26 is capable of performing any of various types of processes by using one or more image processing filters such as a moving average (smoothing) filter, a Gaussian filter, a median filter, a recursive filter, a band-pass filter, and the like.

Further, the image processing circuitry 26 is configured to reconstruct reconstruction data (volume data) from the projection data acquired by performing the rotating imaging process. Further, the image processing circuitry 26 is configured to store the reconstructed volume data into the storage 25. The image processing circuitry 26 is configured to generate a three-dimensional image from the volume data. For example, the image processing circuitry 26 generates a volume rendering image and/or a Multi Planar Reconstruction (MPR) image from the volume data. After that, the image processing circuitry 26 stores the generated three-dimensional image into the storage 25.

The input interface 22 is realized by using a trackball, a switch button, a mouse, and/or a keyboard, used for setting a predetermined region (e.g., a region of interest such as a site of interest); a touchpad used for performing an input operation by touching the operation surface thereof; a touch screen in which a display screen and a touch pad are integrated together; a contactless input circuit using an optical sensor; as well as an audio input circuit; a foot switch used for causing the radiation of X-rays; and/or the like. The input interface 22 is connected to the processing circuitry 21 and is configured to convert an input operation received from an operator into an electrical signal and to output the electrical signal to the processing circuitry 21. Possible examples of the input interface 22 of the present disclosure are not limited to those provided with physical operational component parts such as a mouse, a keyboard, and the like. For example, possible examples of the input interface include an electrical signal processing circuit configured to receive an electrical signal corresponding to an input operation from an external input device provided separately from the apparatus and to output the received electrical signal to a controlling circuit. The display 23 is configured to display a Graphical User Interface (GUI) used for receiving instructions from the operator, as well as any of various types of images generated by the image processing circuitry 26. Further, the display 23 is configured to display any of various types of processing results obtained by the processing circuitry 21.

The processing circuitry 21 is configured to control operations of the entirety of the X-ray diagnosis apparatus 100. More specifically, the processing circuitry 21 is configured to execute various types of processes by reading and executing the program corresponding to the controlling function 211 for controlling the entire apparatus, from the storage 25. For example, the controlling function 211 is configured to control the dose and the turning off and on of the X-rays radiated onto the subject P, by controlling the high voltage generator 11 and adjusting the voltage supplied to the X-ray tube 12, according to an instruction from the operator transferred thereto from the input interface 22. Further, for example, the controlling function 211 is configured to adjust the rotating and the moving of the C-arm 15 and the moving of the tabletop 14 by controlling the C-arm/tabletop mechanism controlling circuitry 19 according to an instruction from the operator. Further, for example, the controlling function 211 is configured to control the radiation range of the X-rays radiated onto the subject P, by controlling the collimator controlling circuitry 20 and adjusting the opening degree of the collimator blades included in the collimator 13, according to an instruction from the operator.

Further, according to an instruction from the operator, the controlling function 211 is configured to control the projection data generating process performed by the image data generating circuitry 24, as well as the image processing processes performed by the image processing circuitry 26, analyzing processes, and the like. Further, the controlling function 211 is configured to exercise control so that the display 23 displays the GUI used for receiving instructions from the operator, any of the images stored in the storage 25, and any of the processing results obtained by the processing circuitry 21. Further, the controlling function 211 is configured to extract one or more puncture route candidate regions in each of which bones, blood vessels, and the like are not included in a puncture needle insertion path (a puncture route) to a site subject to a puncture procedure (a target). This feature will be explained in detail later.

As illustrated in FIG. 1, in addition to the controlling function 211 explained above, the processing circuitry 21 according to the first embodiment is configured to execute the obtaining function 212, the extracting function 213, and the presenting function 214. These functions will be explained in detail later. The processing circuitry 21 is an example of the processing circuitry.

An overall configuration of the X-ray diagnosis apparatus 100 has thus been explained. The X-ray diagnosis apparatus 100 according to the first embodiment configured as described above makes it possible to easily establish a setting for a puncture procedure. More specifically, the X-ray diagnosis apparatus 100 makes it possible to easily establish the setting for the puncture procedure by, when presenting recommended a puncture route, presenting one selected from between one or more candidates obtained by excluding regions positioned outside the imaged range of an image to be referenced during the puncture procedure and one or more candidates positioned within the imaged range, in addition to regions where it is not possible to insert a puncture needle such as blood vessels and bones. In other words, when presenting the recommended puncture routes to a practitioner during a puncture planning process, the X-ray diagnosis apparatus 100 makes it easy to set a puncture route during the puncture planning process, by presenting puncture routes that take into consideration the imaged range to be used during the puncture procedure.

Examples of processes performed by the X-ray diagnosis apparatus 100 will be explained below. In the first embodiment, an example will be explained in which a first image taking apparatus that acquires volume data is the same as a second image taking apparatus to be used when a device is inserted into the subject. In other words, the example will be explained in which a puncture plan is made on the basis of the volume data acquired by the X-ray diagnosis apparatus 100, so that a puncture procedure is performed while an image taken by the X-ray diagnosis apparatus 100 is being referenced. That is to say, the example will be explained in which the volume data is acquired, the puncture plan is made, and the puncture procedure is performed, while the subject P is lying on the tabletop 14.

In that situation, to acquire the volume data, the controlling function 211 controls a rotating imaging process performed for the purpose of realizing a three-dimensional reconstruction. During the rotating imaging process performed for the purpose of realizing the three-dimensional reconstruction, the controlling function 211 exercises control so that projection data is acquired from various directions in a range of approximately 200 degrees around the subject P and so that the volume data is reconstructed through a reconstructing process using the acquired projection data. For example, the controlling function 211 controls the C-arm/tabletop mechanism controlling circuitry 19 so that the projection data is acquired at a predetermined framerate while the C-arm 15 holding the X-ray tube 12 and the X-ray detector 16 are being tuned by approximately 200 degrees. Further, the controlling function 211 controls the image processing circuitry 26 so as to reconstruct the volume data by using the acquired projection data.

After that, the controlling function 211 controls the image processing circuitry 26 so as to generate a three-dimensional image such as a volume rendering image or an MPR image, from the reconstructed volume data. In this situation, the three-dimensional image generated under the control of the controlling function 211 will be displayed on a puncture planning-purpose screen. In other words, the presenting function 214 causes the display 23 to display the puncture planning-purpose screen including the generated three-dimensional image. The practitioner sets a target in the three-dimensional image via the input interface 22, by referencing the three-dimensional image displayed on the display 23.

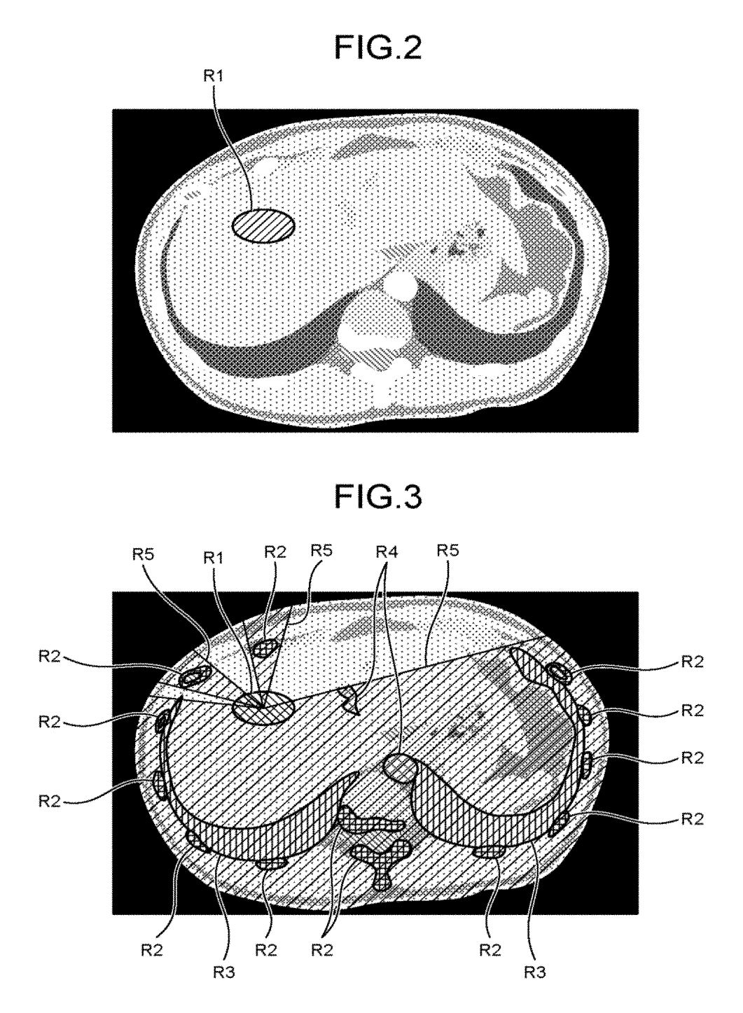

FIG. 2 is a drawing illustrating an example of a target setting process according to the first embodiment. FIG. 2 illustrates an example in which a target is to be set in an MPR image indicating an axial cross-sectional plane of the abdomen. For example, the presenting function 214 causes the MPR image illustrated in FIG. 2 to be displayed on the puncture planning-purpose screen. The practitioner searches for a puncture target (e.g., a tumor in the liver) while manipulating the input interface 22 and changing the displayed cross-sectional plane along the body axis direction and selects an MPR image displaying the target properly. After that, the practitioner sets a target region R1 indicating the target, within the selected MPR image, as illustrated in FIG. 2.

It is possible to transform the position (the coordinates within the image) of the target region R1 set by the practitioner into a coordinate system of the X-ray diagnosis apparatus 100, on the basis of volume data acquisition conditions and the geometry of the X-ray diagnosis apparatus 100. In other words, the controlling function 211 is able to identify the position (the coordinates) of the target region R1 in the coordinate system of the X-ray diagnosis apparatus 100. Further, although FIG. 2 illustrates only the MPR image taken on the axial cross-sectional plane as the image displayed for the puncture planning purpose, possible embodiments are not limited to this example. Another arrangement is also acceptable in which an image taken on another arbitrary cross-sectional plane such as a sagittal cross-sectional plane or a coronal cross-sectional plane is displayed so that a target region is set therein.

Further, on the basis of the volume data, the controlling function 211 sets one or more insertion paths to be used when the device is inserted into the subject. More specifically, on the basis of anatomical position information, the controlling function 211 extracts puncture route candidate regions for the target. Even more specifically, the controlling function 211 extracts the puncture route candidate regions, by identifying sites that are not puncturable (hereinafter, “unpuncturable sites”) from among various sites included in the volume data and excluding the positions of the identified sites. For example, on the basis of anatomical feature points included in the volume data and pixels values, the controlling function 211 identifies the unpuncturable sites such as blood vessels (e.g., the abdominal aorta) and bones in the volume data. After that, the controlling function 211 identifies the regions of the unpuncturable sites within the image in which the target region R1 was set and further extracts the regions other than the identified regions as the puncture route candidate regions.

In this situation, in addition to the unpuncturable regions explained above, the controlling function 211 may exclude, in advance, such regions where the puncture would physically be difficult, from the puncture route candidate regions. More specifically, the controlling function 211 may exclude a region of the subject P positioned on the side that is in contact with the tabletop 14, from the puncture route candidate regions. For example, the controlling function 211 may exclude a region on the dorsal side that is in contact with the tabletop 14, from the puncture route candidate regions.

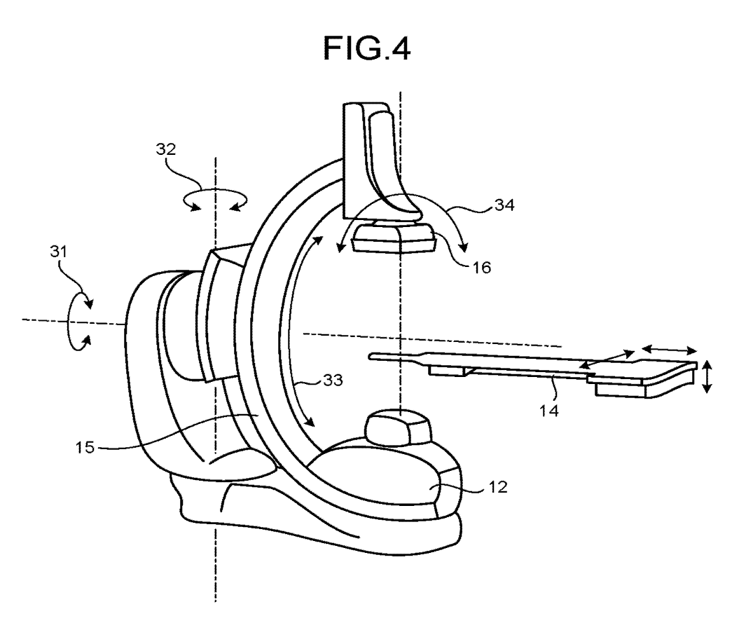

FIG. 3 is a drawing illustrating an example of the process of extracting the puncture route candidate regions performed by the controlling function 211 according to the first embodiment. FIG. 3 illustrates an example in which puncture route candidate regions are extracted with respect to the target region R1 set in FIG. 2. First, the controlling function 211 identifies unpuncturable regions in the image. For example, as illustrated in FIG. 3, the controlling function 211 identifies bone regions R2, lung regions R3, and blood vessel regions R4. Further, among routes from the body surface to the target region R1, the controlling function 211 identifies regions that pass through the identified regions as unpuncturable regions R5. After that, among the routes from the body surface to the target region R1, the controlling function 211 extracts the regions obtained by excluding the identified unpuncturable regions R5 as the puncture route candidate regions.

Returning to the description of FIG. 1, the obtaining function 212 is configured to obtain information about an imaged range to be used during a medical examination by an image taking apparatus that takes a medial image. More specifically, the obtaining function 212 is configured to obtain the information about the imaged range to be used during a puncture procedure by the image taking apparatus that takes the medical image to be referenced during the puncture procedure. Even more specifically, the obtaining function 212 obtains information about movable ranges (moving ranges) and interference ranges of the C-arm 15. For example, as the information about the moving ranges of the C-arm 15, the obtaining function 212 obtains information about a movable limit of the C-arm 15. In other words, the obtaining function 212 obtains information as to from which angles it is possible to take images of the subject P lying on the tabletop 14.

In one example, the obtaining function 212 obtains information about a movable limit of the C-arm 15 illustrated in FIG. 4. FIG. 4 is a drawing for explaining movability of the C-arm 15 according to the first embodiment. Although FIG. 4 illustrates a C-arm of a floor installation type as the C-arm 15, possible embodiments are not limited to this example. The C-arm 15 may be, for example, a C-arm of a type hung from the ceiling. Further, in the present embodiment, the example with a single-plane apparatus is explained; however, possible embodiments are not limited to this example, and a bi-plane apparatus may be used. In that situation, for example, the obtaining function 212 obtains information about moving ranges of both a C-arm and an Q-arm.

For example, as illustrated in FIG. 4, the C-arm 15 is pivotally supported by the supporting unit fixed to the floor, so as to be rotatable in the directions indicated by an arrow 31 while the rotation axis thereof extends in the horizontal direction. In other words, as a result of the C-arm 15 rotating in the directions indicated by the arrow 31, the X-ray tube 12 and the X-ray detector 16 rotate in the directions indicated by an arrow 34. In this situation, the supporting unit is configured so as to be rotatable in the directions indicated by an arrow 32 while the rotation axis thereof extends in the vertical direction. In other words, the C-arm 15 is pivotally supported by the supporting unit so as to be rotatable in the directions indicated by the arrow 32 while the rotation axis thereof extends in the vertical direction. Further, as illustrated in FIG. 4, the C-arm 15 is movable as being slid in the directions indicated by an arrow 33.

As explained above, the C-arm 15 is movable in the various directions. With respect to the C-arm 15 configured in this manner, the obtaining function 212 is configured to obtain the information about the moving range in each of the directions in the coordinate system of the X-ray diagnosis apparatus 100. For example, the obtaining function 212 obtains the information about the moving range of the C-arm 15 in the directions indicated by the arrow 32 in FIG. 4, the moving range of the C-arm 15 in the directions indicated by the arrow 33, and the moving range of the C-arm 15 in the directions indicated by the arrow 34. In other words, the obtaining function 212 obtains the information about the movable limits of the C-arm 15 in the various directions. Further, the obtaining function 212 is also capable of obtaining information about moving ranges of the tabletop 14 in addition to the moving ranges of the C-arm 15. For example, as illustrated in FIG. 4, the obtaining function 212 is capable of obtaining information about a moving range of the tabletop 14 in the lengthwise directions, a moving range thereof in the widthwise directions, and a moving range thereof in the vertical directions.

Further, the obtaining function 212 obtains information about interference ranges of the C-arm 15. More specifically, the obtaining function 212 obtains the information about interference between the C-arm 15 and the tabletop 14 and interference between the C-arm 15 and the subject P that may occur while an X-ray image (e.g., a fluoroscopic image) to be referenced during a puncture procedure is being acquired. For example, with respect to an image taking condition used during the puncture procedure, the obtaining function 212 obtains a range in which the C-arm 15 and the tabletop 14 interfere with each other and a range in which the C-arm 15 and the subject P interface with each other, in the coordinate system of the X-ray diagnosis apparatus 100. In this situation, it is possible to judge whether or not the C-arm 15 and the tabletop 14 interfere with each other on the basis of, for example, the geometry of the X-ray diagnosis apparatus 100. Further, it is possible to judge whether or not the C-arm 15 and the subject P interfere with each other, for example, by arranging a model based on the physique of the subject P into the coordinate system of the X-ray diagnosis apparatus 100 and checking to see whether or not the model and the C-arm 15 interfere with each other.

In this situation, the information about the interference ranges obtained by the obtaining function 212 indicates interference ranges under the image taking condition used during the puncture procedure. In other words, the obtaining function 212 obtains the interference ranges corresponding to the situation where the subject P is arranged in the position where the subject P will be during the puncture procedure. As explained above, to perform the puncture procedure, the X-ray diagnosis apparatus 100 performs the rotating imaging process to reconstruct the volume data and subsequently acquires the X-ray image during the puncture procedure. In this situation, depending on the position of the puncture target, the position of the subject P may be moved for the purpose of acquiring an optimal X-ray image. More specifically, to arrange the target in the vicinity of the rotation center (the isocenter) of the C-arm 15, the tabletop 14 may be moved in some situations. In those situations, the obtaining function 212 obtains interference ranges corresponding to the state after the tabletop 14 is moved.

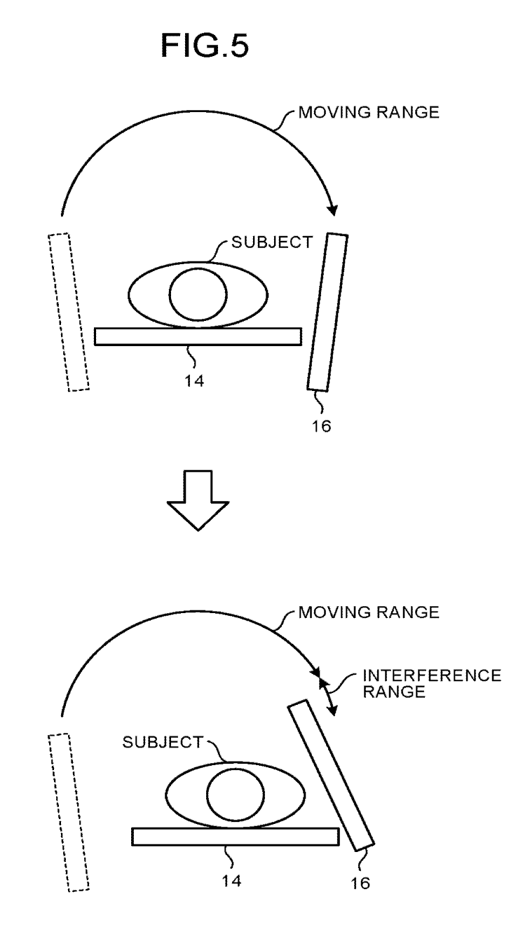

FIG. 5 is a drawing illustrating an example of an interference range obtained by the obtaining function 212 according to the first embodiment. FIG. 5 illustrates an example in which the C-arm 15 is rotated while the rotation axis thereof extends in the body axis direction, for the subject P lying on the tabletop 14. Further, although FIG. 5 illustrates only the X-ray detector 16, the X-ray tube 12 is, in actuality, arranged in a position so as to oppose the X-ray detector 16. For example, the rotating imaging process is performed with the positional relationship illustrated in the top section of FIG. 5, and when a target has been set on a tumor in the liver, the controlling function 211 exercises control so as to move the tabletop 14 in such a manner that the position of the set target is arranged at the isocenter. For example, the controlling function 211 moves the tabletop 14 on which the subject P is lying, in the manner illustrated in the bottom section of FIG. 5.

When the tabletop 14 has been moved as explained above, the obtaining function 212 obtains an interference range corresponding to the state after the tabletop 14 is moved. In other words, out of the moving range (the moving range indicated in the top section of FIG. 5) of the C-arm 15 corresponding to the state before the tabletop 14 is moved, the obtaining function 212 obtains the interference range (the interference range indicated in the bottom section of FIG. 5) that is caused by the moving of the tabletop 14.

Further, in addition to the interference explained above, the obtaining function 212 is also capable of obtaining information about interference between the practitioner and the C-arm 15 and interference between the puncture needle and the C-arm 15. For example, the obtaining function 212 may estimate the position of the practitioner with respect to the subject P on the basis of the position of the set target and obtain information about interference between the estimated position of the practitioner and the C-arm 15. Further, the obtaining function 212 may estimate the position of the puncture needle on the basis of the estimated position of the practitioner and obtain information about interference between the estimated position of the puncture needle and the C-arm 15. In this situation, the position of the practitioner based on the position of the target is estimated by using information about positions of the practitioner stored in the storage 25 in advance. The information about the positions of the practitioner is information in which each of various positions of the practitioner with respect to the subject P is kept in correspondence with a different one of positions of the target (various sites and roughly specified positions in each of the sites).

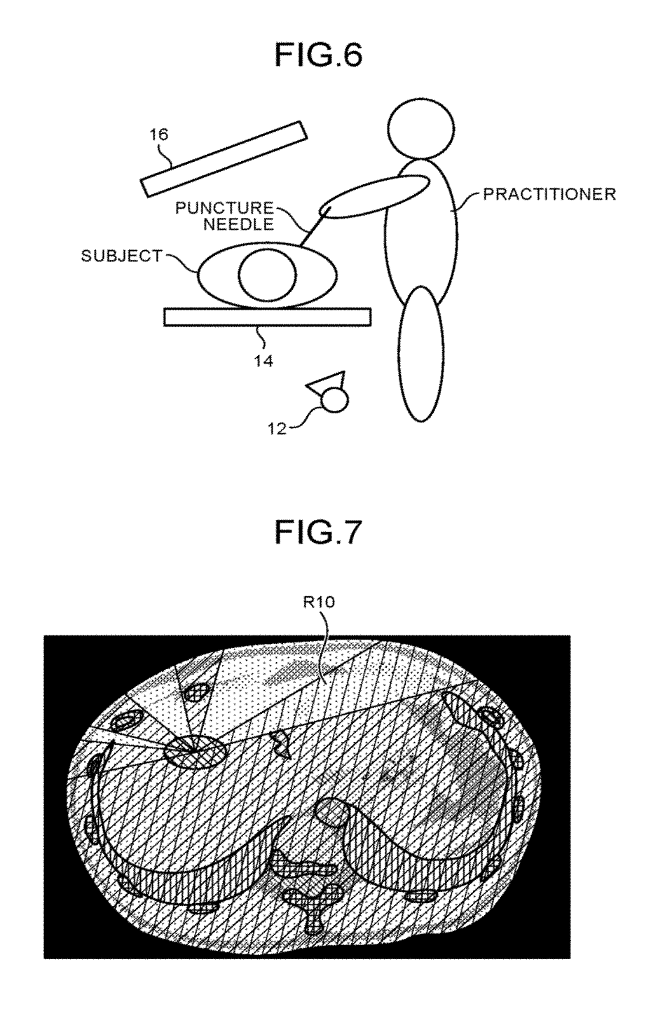

FIG. 6 is a drawing for explaining an example of the information about the interference obtained by the obtaining function 212 according to the first embodiment. For example, on the basis of the position of the set target, the obtaining function 212 estimates the position of the practitioner with respect to the subject P and the position of the puncture needle as illustrated in FIG. 6 and further obtains interference ranges on the basis of the estimated positions. In the example above, the position of the practitioner with respect to the subject P and the position of the puncture needle are estimated on the basis of the position of the target; however, possible embodiments are not limited to this example. For instance, on the basis of the puncture route candidate regions (the candidate regions extracted on the basis of the anatomical position information) extracted by the controlling function 211, the position of the practitioner and the position of the puncture needle may be estimated. In that situation, for example, on the basis of the puncture route candidate regions extracted by the controlling function 211, the obtaining function 212 estimates the insertion direction of the puncture needle for the subject P and further estimates the position of the practitioner and the position of the puncture needle on the basis of the estimated insertion direction.

Returning to the description of FIG. 1, the extracting function 213 is configured to evaluate the puncture route candidate regions, by comparing the insertion paths to the target subject to the puncture procedure with the imaged range. More specifically, the extracting function 213 is configured to extract, on the basis of the information about the imaged range, one selected from between at least one insertion path positioned outside the imaged range and at least one insertion path positioned within the imaged range, from insertion paths to the target subject to the puncture procedure. Even more specifically, from within the puncture route candidate regions extracted on the basis of the anatomical position information, the extracting function 213 extracts one selected from between at least one insertion path positioned outside the imaged range and at least one insertion path positioned within the imaged range. For example, on the basis of the information about the movable ranges and the interference ranges of the C-arm 15, the extracting function 213 extracts one selected from between at least one insertion path positioned outside the imaged range and at least one insertion path positioned within the imaged range, from the puncture route candidate regions for the target. In other words, on the basis of the information about the moving ranges and the interference ranges of the C-arm 15 obtained by the obtaining function 212, the extracting function 213 identifies, within the coordinate system of the X-ray diagnosis apparatus 100, a range of angles at which either it will be difficult or it will be possible to acquire an optimal X-ray image as the image to be referenced during the puncture procedure. Further, from the puncture route candidate regions extracted by the controlling function 211, the extracting function 213 extracts one or more regions in each of which the X-ray image acquired in the identified range will be an optimal X-ray image referenced during the puncture procedure.

In this situation, as the image to be referenced during the puncture procedure, for example, two fluoroscopic images may be acquired, namely, an image in a direction parallel to the insertion direction of the puncture needle and another image in a direction capturing the long axis of the puncture needle. In other words, examples of the images to be referenced during the puncture procedure include: the image acquired while controlling the X-ray radiation direction so as to be parallel to the longitudinal direction of the puncture needle (i.e., an image in which the puncture needle is rendered therein as a point); and the other image acquired while controlling the X-ray radiation direction so as to be orthogonal to the longitudinal direction of the puncture needle (i.e., an image in which the puncture needle is rendered therein as a line). Accordingly, during the puncture procedure, the C-arm 15 is arranged in such a position where such X-ray images can be acquired. On the basis of the information about the moving ranges and the information about the interference ranges of the C-arm 15 obtained by the obtaining function 212, the extracting function 213 judges whether or not it is possible to arrange the C-arm 15 in such a position, for each of the puncture routes included in the puncture route candidate regions.

In other words, with respect to each of the puncture routes included in the puncture route candidate regions, the extracting function 213 judges whether or not the position of the C-arm 15 corresponding to the time when the fluoroscopic images are acquired is included in the moving ranges and whether or not the same is included in the interference ranges. In the following sections, an example will be explained in which the extracting function 213 extracts puncture routes positioned outside the imaged range. For example, the extracting function 213 extracts, as the routes positioned outside the imaged range, such puncture routes with which the position of the C-arm 15 corresponding to the time when the fluoroscopic images are acquired are not included in the moving ranges and such puncture routes with which the position of the C-arm 15 corresponding to the time when the fluoroscopic images are acquired are included in the interference ranges. The extracting function 213 extracts the regions positioned outside the imaged range from the puncture route candidate regions, by performing the abovementioned judgment process with respect to each of the puncture routes included in the puncture route candidate regions.

After that, the extracting function 213 extracts one or more puncturable regions obtained by excluding the regions positioned outside the imaged range from the puncture route candidate regions. FIG. 7 is a drawing illustrating examples of regions extracted by the extracting function 213 according to the first embodiment. For example, as illustrated in FIG. 7, from the puncture route candidate regions, the extracting function 213 extracts the region positioned outside the imaged range. After that, as a region that is not subject to the puncture procedure, the extracting function 213 extracts a region R10 including the extracted region and the unpuncturable regions (the regions R5 in FIG. 3) extracted by the controlling function 211. In other words, the extracting function 213 extracts the puncturable region that takes into consideration the anatomical position information and the imaged range. In the embodiment above, the example is explained in which the region positioned outside the imaged range is extracted from the puncture route candidate regions, on the basis of the moving ranges and the interference ranges of the C-arm 15. However, possible embodiments are not limited to this example. When there is no interference range, the region positioned outside the imaged range may be extracted from the puncture route candidate regions, only on the basis of the moving ranges of the C-arm 15.

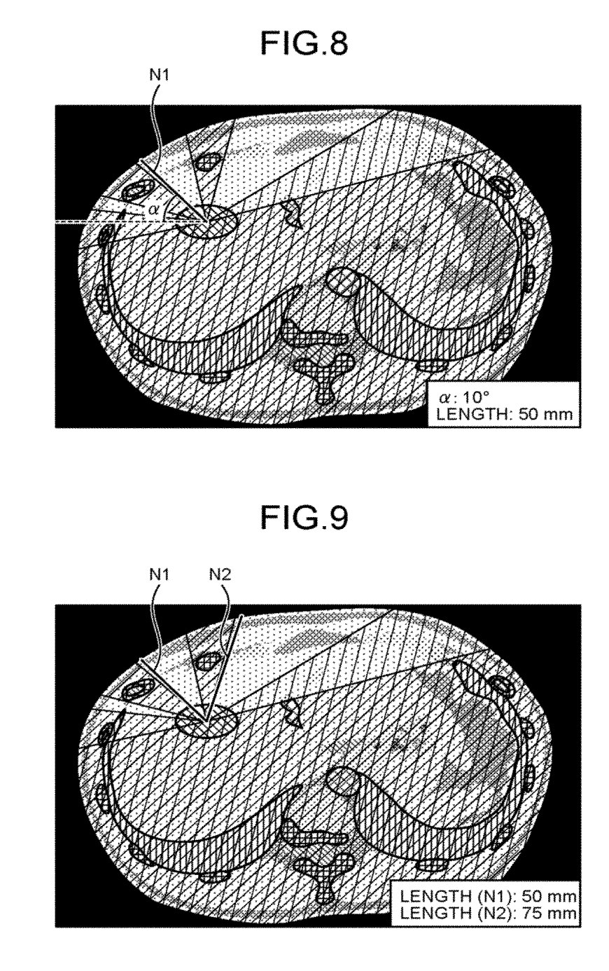

Returning to the description of FIG. 1, the presenting function 214 is configured to present a result of the evaluation. More specifically, the presenting function 214 is configured to present, from among the insertion paths to the target, one selected from between at least one insertion path obtained by excluding the insertion paths positioned outside the imaged range and at least one insertion path positioned within the imaged range. FIG. 8 is a drawing illustrating an example of a display presented on the display 23 by the presenting function 214 according to the first embodiment. For example, as illustrated in FIG. 8, the presenting function 214 displays, on a puncture planning-purpose screen, an image in which it is possible to identify the regions that are not subject to the puncture procedure and the puncturable regions.

In this situation, in addition to the puncturable regions, the presenting function 214 is also capable of presenting a recommended puncture route. For example, as illustrated in FIG. 8, the presenting function 214 displays, on the puncture planning-purpose screen, a recommended puncture route N1. In this situation, as illustrated in FIG. 8, the presenting function 214 may display “Length: 50 mm” indicating the distance from the body surface to the center of the target on the recommended puncture route N1 and/or “α: 10 degrees” indicating the angle of the puncture needle with respect to the horizontal direction, together with the recommended puncture route N1.

In this situation, the presenting function 214 extracts the recommended puncture route from the puncturable regions, on the basis of the distance from the body surface to the center of the target, the insertion angle of the puncture needle, the type of the puncture needle, and the like. For example, among the routes in the puncturable regions, the presenting function 214 extracts such a route that has the shortest distance from the body surface to the center of the target, as a recommended puncture route. Alternatively, among the routes in the puncturable regions, the presenting function 214 may extract such a route that is positioned closest to the vertical direction (e.g., such a route of which the insertion angle with respect to the horizontal direction is closest to 90 degrees), as a recommended puncture route. In another example, the presenting function 214 may extract a recommended puncture route in accordance with types of puncture needle used for various manipulations such as biopsy, ablation, cryotherapy, and the like.

The recommended puncture route may arbitrarily be extracted on the basis of any of the various types of conditions described above. For example, it is acceptable to set priority levels among the distance from the body surface to the center of the target, the insertion angle of the puncture needle, and the type of the puncture needle, so as to extract a recommended puncture route in such a manner that the conditions are satisfied in the descending order of the priority levels. Alternatively, it is also acceptable to extract a recommended puncture route by evaluating the conditions described above in a composite manner.

In the embodiment described above, the example is explained in which a single puncture needle is used; however, possible embodiments are not limited to this example. Even when a plurality of puncture needles is used, it is also possible to similarly present a recommended puncture route. FIG. 9 is a drawing illustrating another example of a display presented on the display 23 by the presenting function 214 according to the first embodiment. For example, as illustrated in FIG. 9, the presenting function 214 displays on the puncture planning-purpose screen, recommended puncture routes N1 and N2 for two puncture needles. In this situation, in addition to the extraction conditions for the recommended puncture route described above, the presenting function 214 extracts the recommended puncture routes N1 and N2, by taking into consideration the positional relationship between the two puncture needles. For example, when a manipulation is performed while using two or more puncture needles, the positional arrangements of the puncture needles with respect to a tumor change in accordance with the size and the shape of the tumor. For this reason, the presenting function 214 extracts, from the puncturable regions, a number of sets each made up of routes satisfying the positional relationship in which the plurality of puncture needles can be arranged and further extracts one of the extracted sets of routes that satisfies the extraction conditions described above as recommended puncture routes.

Further, when presenting the recommended puncture routes for the plurality of puncture needles, the presenting function 214 is also able to display, together therewith, information indicating the distances from the body surface to the center of the target and/or the angles of the puncture needles with respect to the horizontal direction. For example, as illustrated in FIG. 9, the presenting function 214 may display, on the puncture planning-purpose screen, “Length (N1): 50 mm” indicating the distance from the body surface to the center of the target for the puncture route N1 and “Length (N2): 75 mm” indicating the distance from the body surface to the center of the target for the puncture route N2.

After presenting the recommended puncture route, the X-ray diagnosis apparatus 100 receives an operation to confirm a puncture route. For example, the X-ray diagnosis apparatus 100 receives a confirming operation to accept the presented recommended puncture route or a confirming operation to correct the puncture route and confirm the corrected puncture route as the route.

As explained above, the X-ray diagnosis apparatus 100 makes it possible to easily establish the setting for the puncture procedure by, when presenting the recommended puncture route, presenting such a puncture route that takes into consideration the imaged range to be used during the puncture procedure, in addition to the information about the unpuncturable sites such as blood vessels, bones, the lungs, and the like. In this situation, the X-ray diagnosis apparatus 100 is further capable of correcting the recommended puncture route and changing the X-ray image acquisition condition to be used during the puncture procedure, in accordance with whether a metal artifact is present or not.

When the puncture procedure is performed while X-rays are being radiated, a metal artifact may occur in some situations, depending on the directions of the puncture route and the X-ray beam, as well as the type of the puncture needle. For example, when a manipulation is performed by using a thick puncture needle, while the insertion direction of the puncture needle is substantially parallel to the direction of the X-ray beam, a metal artifact may occur, in some situations, from the tip end of the puncture needle in the forward direction. In those situations, because the metal artifact occurs along the insertion direction of the puncture needle, i.e., the direction toward the target, the visibility of the fluoroscopic image to be referenced will be degraded.

To address those situations, the X-ray diagnosis apparatus 100 exercise control so as to change either the puncture route or the X-ray projection direction. For example, the presenting function 214 judges whether or not a metal artifact would occur, on the basis of the insertion direction of the puncture needle for the extracted recommended puncture route and the acquisition direction of the fluoroscopic image. Further, when it is determined that a metal artifact would occur, the presenting function 214 displays, on the puncture planning-purpose screen, information indicating that there is a possibility that a metal artifact may occur and an alternative puncture route. Accordingly, the practitioner is able to select one from between the recommended puncture route and the alternative puncture route. Consequently, the practitioner is able to execute the puncture procedure while referencing a fluoroscopic image having higher visibility.

Further, when it is determined that a metal artifact would occur, the presenting function 214 displays, on the puncture planning-purpose screen, information indicating that there is a possibility that a metal artifact may occur and a GUI used for receiving a confirming operation indicating whether the X-ray projection direction should be shifted. When having received a confirming operation indicating that the X-ray projection direction should be shifted, the controlling function 211 exercises control so as to shift the X-ray projection direction into such a direction that will cause no metal artifact. Consequently, the practitioner is able to execute the puncture procedure while referencing a fluoroscopic image having higher visibility.

In the explanations above, the examples are explained in which, when it is determined that a metal artifact would occur with the recommended puncture route, the alternative puncture route is displayed or the GUI is displayed to receive the confirming operation to indicate whether or not the X-ray projection direction should be shifted. However, possible embodiments are not limited to these examples. For instance, another arrangement is also acceptable in which it is judged in advance whether or not a metal artifact will occur, so that a route that will cause no metal artifact is extracted as a recommended puncture route.

Further, the X-ray diagnosis apparatus 100 is also capable of automatically applying a metal artifact reducing process to the metal artifact. For example, the controlling function 211 exercises control so as to reduce the metal artifact occurring in the fluoroscopic image.

Further, when it is impossible to extract a puncturable region, the X-ray diagnosis apparatus 100 is also capable of presenting a suggestion that the image taking condition should be changed. For example, when the extracting function 213 has extracted no puncturable region, the presenting function 214 may present information suggesting to the practitioner that the image taking condition of the fluoroscopic image to be acquired during the puncture procedure should be changed (e.g., by enlarging the size of the field of view).

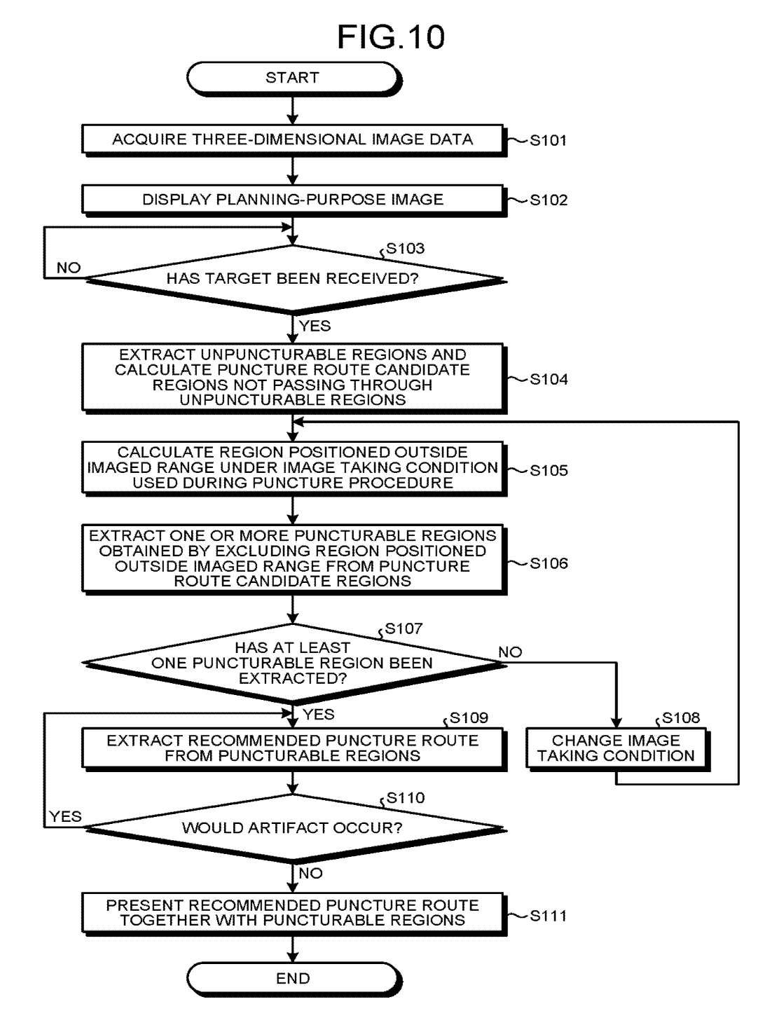

Next, a process performed by the X-ray diagnosis apparatus 100 according to the first embodiment will be explained, with reference to FIG. 10. FIG. 10 is a flowchart illustrating a processing procedure of the X-ray diagnosis apparatus 100 according to the first embodiment. Steps S101, S103, S104, and S108 illustrated in FIG. 10 are steps realized as a result of the processing circuitry 21 reading and executing the program corresponding to the controlling function 211 from the storage 25. Steps S102 and S109 to S111 are steps realized as a result of the processing circuitry 21 reading and executing the program corresponding to the presenting function 214 from the storage 25. Step S105 is a step realized as a result of the processing circuitry 21 reading and executing the programs corresponding to the obtaining function 212 and the extracting function 213 from the storage 25. Steps S106 and S107 are steps realized as a result of the processing circuitry 21 reading and executing the program corresponding to the extracting function 213 from the storage 25.

At step S101, the processing circuitry 21 acquires three-dimensional image data (volume data). At step S102, the processing circuitry 21 generates and displays an image used for a puncture planning purpose. At step S103, the processing circuitry 21 judges whether or not a target has been received. When a target has been received (step S103: Yes), the processing circuitry 21 extracts, at step S104, unpuncturable regions on the basis of the anatomical position information in the volume data and calculates puncture route candidate regions that do not go through the unpuncturable regions. On the contrary, the processing circuitry 21 is in a standby state until a target is received (step S103: No).

At step S105, the processing circuitry 21 calculates a region positioned outside the imaged range under the image taking condition to be used during the puncture procedure. More specifically, on the basis of the moving ranges and the interference ranges of the C-arm 15, the processing circuitry 21 extracts the region positioned outside the imaged range, from the puncture route candidate regions. At step S106, the processing circuitry 21 extracts one or more puncturable regions obtained by excluding the region positioned outside the imaged range from the puncture route candidate regions. At step S107, the processing circuitry 21 judges whether or not at least one puncturable region has been extracted.

In this situation, when at least one puncturable region has been extracted (step S107: Yes), the processing circuitry 21 extracts a recommended puncture route from the puncturable regions at step S109. On the contrary, when no puncturable region has been extracted (step S107: No), the processing circuitry 21 changes the image taking condition to be used during the puncture procedure at step S108. The process then returns to step S105 where the processing circuitry 21 calculates a region positioned outside the imaged range.

At step S110, the processing circuitry 21 judges whether or not an artifact would occur. When it is determined that an artifact would occur (step S110: Yes), the process returns to step S109 where the processing circuitry 21 re-extracts another recommended puncture route. On the contrary, when it is determined that no artifact will occur (step S110: No), the processing circuitry 21 presents the recommended puncture route together with the puncturable regions at step S111.

As explained above, according to the first embodiment, the obtaining function 212 is configured to obtain the information about the imaged range to be used during the medical examination by the image taking apparatus that takes the medical image. On the basis of the information about the imaged range, the extracting function 213 is configured to extract the one selected from between at least one insertion path positioned outside the imaged range and at least one insertion path positioned within the imaged range, from the insertion paths to the target site subject to the puncture procedure. From among the insertion paths to the target site, the presenting function 214 is configured to present one selected from between at least one insertion path obtained by excluding the insertion paths positioned outside the imaged range and at least one insertion path positioned within the imaged range. Accordingly, the X-ray diagnosis apparatus 100 according to the first embodiment is able to present the puncture route while taking into consideration the imaged range to be used during the puncture procedure and thus makes it possible to easily establish a setting for the puncture procedure.

Further, according to the first embodiment, from the puncture route candidate regions extracted on the basis of the anatomical position information, the extracting function 213 is configured to extract one selected from between at least one insertion path positioned outside the imaged range and at least one insertion path positioned within the imaged range. Accordingly, the X-ray diagnosis apparatus 100 according to the first embodiment is able to simultaneously present the puncture routes that include no unpuncturable sites and thus makes it possible to more easily establish a setting for the puncture procedure.

Further, according to the first embodiment, the obtaining function 212 is configured to obtain the information about the movable ranges and the interference ranges of the C-arm 15. On the basis of the information about the movable ranges and the interference ranges of the C-arm 15, the extracting function 213 is configured to extract one selected from between the insertion paths positioned outside the imaged range and the insertion paths positioned within the imaged range, from the insertion paths to the target site. Consequently, the X-ray diagnosis apparatus 100 according to the first embodiment is able to present the puncture routes while taking into consideration the movable ranges and the interference ranges of the C-arm 15 during the puncture procedure.

Further, according to the first embodiment, the obtaining function 212 is configured to obtain the information about the position, during the puncture procedure, of the practitioner who performs the puncture procedure on the subject. By using the information about the position of the practitioner during the puncture procedure in addition to the information about the imaged range, the extracting function 213 is configured to extract one selected from between the insertion paths positioned outside the imaged range and the insertion paths positioned within the imaged range, from the insertion paths to the target site. Consequently, the X-ray diagnosis apparatus 100 according to the first embodiment is able to present the puncture routes while taking into consideration the interference with the practitioner.

Further, according to the first embodiment, the presenting function 214 is configured to present, in an identifiable manner, the puncture route candidate regions for the target, together with the one selected from between the insertion paths positioned outside the imaged range and the insertion paths positioned within the imaged range. Consequently, the X-ray diagnosis apparatus 100 according to the first embodiment enables the viewer to recognize, at a glance, such regions within the puncture route candidate regions that are not subject to the puncture procedure and thus makes it possible to easily correct the recommended puncture route.

### Second Embodiment

The first embodiment has thus been explained. It is possible to carry out the present disclosure in various different modes other than those explained in the first embodiment.

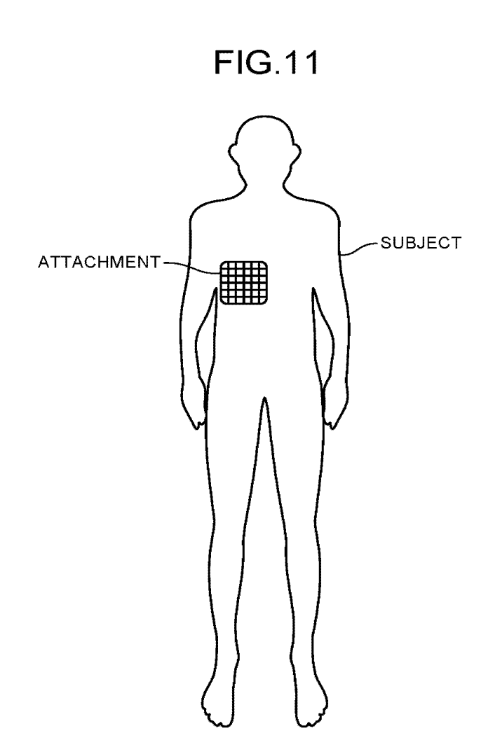

In the first embodiment described above, the example is explained in which the regions obtained by excluding the region positioned outside the imaged range during the puncture procedure from the puncture route candidate regions are extracted as the puncturable regions; however possible embodiments are not limited to this example. For instance, position information of an attachment device (hereinafter, “attachment”) attached to the subject P may further be used. FIG. 11 is a drawing illustrating an example of the attachment according to the second embodiment. To perform the puncture procedure, for example, an attachment indicating a coordinate system such as that illustrated in FIG. 11 is attached to the subject P. The attachment is configured to indicate the puncture insertion point determined at the time of making a puncture plan, within the coordinate system. In other words, the puncture insertion point is set within the region in which the attachment is attached. Accordingly, the extracting function 213 may be configured so as to identify the region where the attachment is attached to the body surface out of the puncturable regions and to further extract a recommended puncture route from within the identified region.

Further, in the embodiment described above, the example is explained in which the puncture procedure is performed while the fluoroscopy is being executed by the X-ray diagnosis apparatus 100; however, possible embodiments are not limited to this example. For instance, the puncture procedure may be performed while a medical image is being taken in another modality. For example, when the puncture procedure is performed while a medical image is being taken by an X-ray Computed Tomography (CT) apparatus, processing circuitry provided for the X-ray CT apparatus performs the same processes as those performed by the obtaining function 212, the extracting function 213, and the presenting function 214 described above.

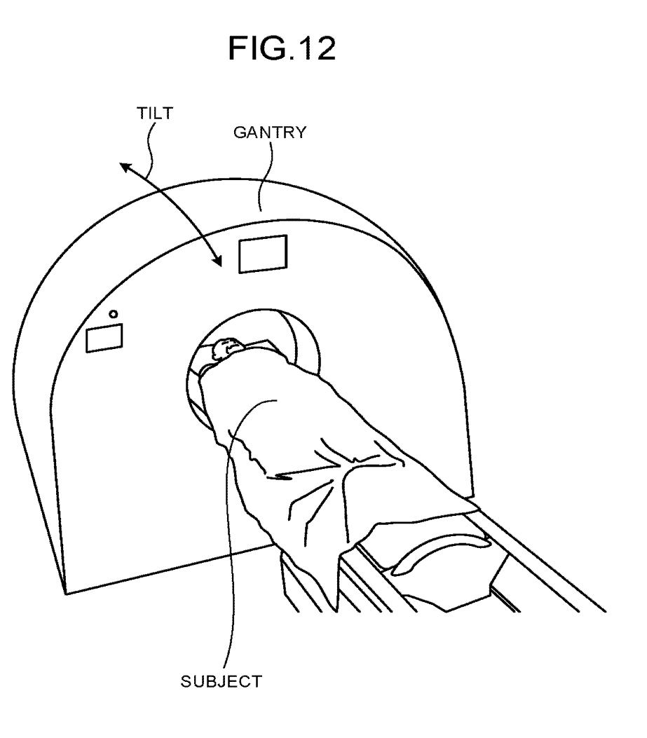

In this situation, the X-ray CT apparatus includes a gantry that is tiltable at a predetermined tilting angle with respect to the subject P. Further, an obtaining function provided for the X-ray CT apparatus is configured to obtain information about the tilting angle of the gantry, as information about an imaged range. FIG. 12 is a drawing for explaining an example of the tilting angle of the gantry included in the X-ray CT apparatus according to the second embodiment. For example, as illustrated in FIG. 12, in the X-ray CT apparatus according to the second embodiment, it is possible to tilt the gantry at the predetermined angle. With the X-ray CT apparatus configured in this manner, when the liver is to be punctured while avoiding the lungs, for example, the puncture procedure is performed while acquiring an image by tilting the gantry at a tilting angle in the range of approximately 25 degrees to 30 degrees with respect to the subject P.

The obtaining function provided for the X-ray CT apparatus is configured to obtain the range of angles at which the gantry is tiltable. Further, from the puncture route candidate regions extracted by a controlling function, an extracting function is configured to extract one or more regions for which the tilting angle used for the image acquisition during the puncture procedure will be outside the range. Further, the extracting function is configured to extract one or more puncturable regions by excluding the one or more extracted regions from the puncture route candidate regions.

Further, when the puncture procedure is performed while a medical image is being taken by an ultrasound diagnosis apparatus, processing circuitry provided for the ultrasound diagnosis apparatus performs the same processes as those performed by the obtaining function 212, the extracting function 213, and the presenting function 214 described above. In this situation, the ultrasound diagnosis apparatus includes an ultrasound probe configured to transmit and receive ultrasound waves to and from the subject P. Also, a puncture needle adaptor configured to guide a puncture needle is attached to the ultrasound probe. Further, an obtaining function provided for the ultrasound diagnosis apparatus is configured to obtain information about a transmission and reception state of the ultrasound waves to and from the target, as information about an imaged range.

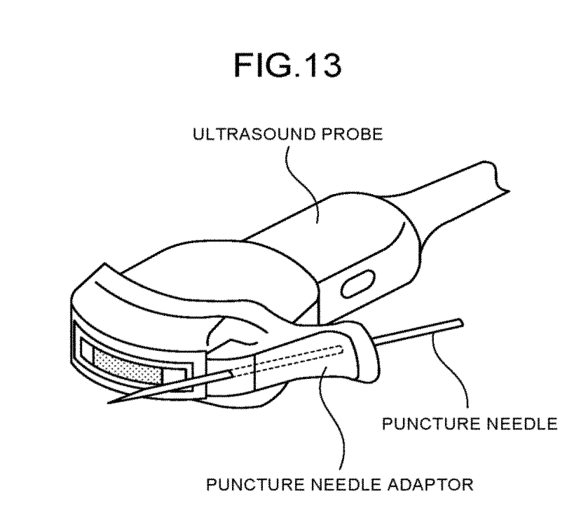

While an ultrasound diagnosis apparatuses is being used, when an artifact caused by gas or bones is included in ultrasound image data, the visibility of the referenced image will be degraded. To cope with this situation, on the basis of the positional relationship between the ultrasound wave transmitting and receiving surface of the ultrasound probe and the puncture needle adaptor, the obtaining function is configured to obtain information about the sites in the subject's body that are to transmit and receive ultrasound waves, for each of the routes included in the puncture route candidate regions. FIG. 13 is a drawing illustrating an example of the ultrasound probe according to the second embodiment. For example, as illustrated in FIG. 13, the ultrasound probe has attached thereto a puncture needle adaptor configured to guide the insertion of a puncture needle. In this situation, the attached position of the puncture needle adaptor to the ultrasound probe is determined for each ultrasound probe (or for each puncture needle adaptor), so that it is possible to uniquely identify the positional relationship between the ultrasound waves transmitting and receiving surface of the ultrasound probe and the puncture needle adaptor, in accordance with the attached position.

Consequently, the obtaining function provided for the ultrasound diagnosis apparatus is configured to obtain the type of the ultrasound probe and the type of the puncture needle adaptor being used and to further identify the positional relationship between the ultrasound wave transmitting and receiving surface and the puncture needle adaptor on the basis of the obtained information about the types. After that, on the basis of the identified positional relationship, the obtaining function is configured to obtain the information about the sites in the subject P's body that are to transmit and receive ultrasound waves, for each of the routes included in the puncture route candidate regions. For example, the obtaining function obtains information indicating whether or not a lung or a bone is included in the region to and from which an ultrasound wave is to be transmitted and received, when the puncture needle is inserted through a certain route included in the puncture route candidate regions. The obtaining function is configured to obtain the abovementioned information for each of the routes included in the puncture route candidate regions.

On the basis of the information about the transmission and reception state of the ultrasound waves to and from the target, an extracting function provided for the ultrasound diagnosis apparatus is configured to extract one or more insertion paths positioned outside the imaged range, from insertion paths to the target. In other words, the extracting function is configured to extract such regions in which the ultrasound wave transmission destination includes a lung or a bone, from the puncture route candidate regions extracted by a controlling function. Further, the extracting function is configured to extract one or more puncturable regions by excluding the regions in which the ultrasound wave transmission destination includes a lung or a bone, from the puncture route candidate regions.

Further, in the embodiments described above, the example is explained in which the modality used for acquiring the volume data is the same as the modality used for acquiring the image referenced during the puncture procedure; however, possible embodiments are not limited to this example. Another arrangement is also acceptable in which the modality used for acquiring the volume data is different front the modality used for acquiring the image referenced during the puncture procedure. In that situation, a position aligning process is performed between the coordinate system of the volume data and the coordinate system of the modality used for acquiring the image referenced during the puncture procedure, so that the processes described above are performed after the position aligning process. To perform the position aligning process on the coordinate systems, it is possible to use an arbitrary one of existing position aligning methods.

Further, in the embodiments described above, the example is explained in which it is judged whether or not a metal artifact would occur before the image referenced during the puncture procedure is acquired. However, possible embodiments are not limited to this example. For instance, another arrangement is acceptable in which the image referenced during the puncture procedure is acquired so that the processes are performed on the basis of an artifact included in the acquired image. In that situation, for example, when the artifact included in the acquired image is different from an artifact expected in advance, the controlling function 211 exercises control so as to change the image taking condition.

Further, in the embodiments described above, the example is explained in which the puncturable regions are extracted, by excluding the region positioned outside the imaged range from the puncture route candidate regions. However, possible embodiments are not limited to this example. For instance, puncturable regions may be extracted on the basis of restrictions specific to the facility or restrictions specific to the practitioner. In this situation, examples of the restrictions specific to the facility include information about the apparatus installed in the facility (e.g., the type of the gantry included in the X-ray CT apparatus) and the type of the puncture needle being used. Examples of the restrictions specific to the practitioner include locations and directions (e.g., the difference in the manipulation direction depending on the practitioner's dominant hand) in which it is easier for the practitioner to perform manipulations due to his/her experience. For example, the storage may store therein, in advance, the restrictions specific to the facility and the restrictions specific to the practitioner. Further, when extracting puncturable regions, the extracting function extracts the puncturable regions by reading the stored information. In one example, after extracting the puncturable regions on the basis of the imaged range as explained above, the extracting function may further exercise control so as to limit the puncturable regions on the basis of the restrictions specific to the facility and/or the restrictions specific to the practitioner.

Further, in the embodiments described above, the example is explained in which the apparatus configured to extract the puncturable regions extracts the puncture route candidate regions; however, possible embodiments are not limited to this example. For instance, the X-ray diagnosis apparatus 100 may extract puncturable regions by using puncture route candidate regions extracted by another apparatus.

Further, in the embodiments described above, the example is explained in which the puncturable regions are extracted by extracting the puncture routes positioned outside the imaged range from the puncture route candidate regions; however, possible embodiments are not limited to this example. For instance, another arrangement is acceptable in which puncturable regions are extracted by extracting puncture routes that are positioned within the imaged range from the puncture route candidate regions.

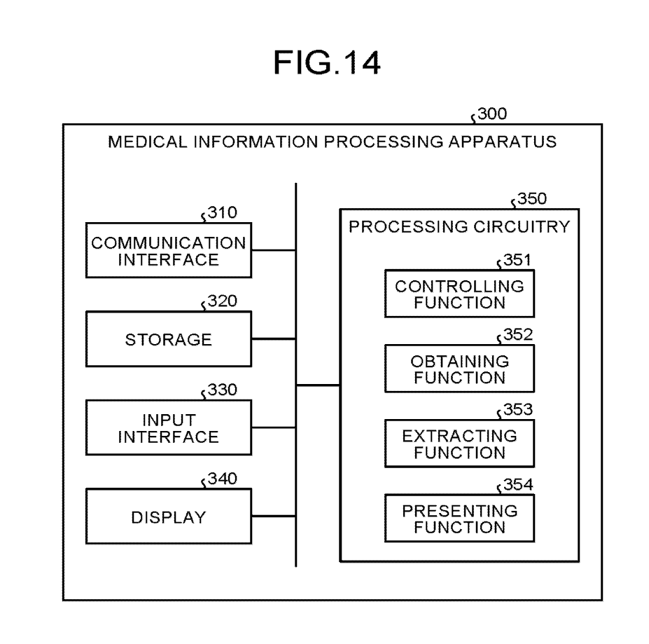

Further, in the embodiments described above, the example is explained in which a medical image diagnosis apparatus (e.g., the X-ray diagnosis apparatus 100) performs the various types of processes; however, possible embodiments are not limited to this example. For instance, an image information processing apparatus realized by using a workstation or the like may perform the various types of processes. FIG. 14 is a diagram illustrating an exemplary configuration of a medical information processing apparatus 300 according to the second embodiment.

For example, as illustrated in FIG. 14, the medical information processing apparatus 300 includes a communication interface 310, storage 320, an input interface 330, a display 340, and processing circuitry 350.

The communication interface 310 is connected to the processing circuitry 350 and is configured to control the transfer of various types of data and the communication between any of various types of X-ray diagnosis apparatuses 100 and/or apparatuses of other modalities that are connected to one another via a network. For example, the communication interface 310 is realized by using a network card, a network adaptor, a Network Interface Controller (NIC), or the like. In the second embodiment, the communication interface 310 is configured to receive the volume data from the X-ray diagnosis apparatus 100 or an apparatus of another modality and to output the received volume data to the processing circuitry 350. Further, the communication interface 310 is configured to receive information about the imaged range to be used during the puncture procedure from the apparatus in the other modality that acquires the image during the puncture procedure and to output the received information about the imaged range to be used during the puncture procedure to the processing circuitry 350.

The storage 320 is connected to the processing circuitry 350 and is configured to store therein various types of data. For example, the storage 320 is realized by using a semiconductor memory element such as a Random Access Memory (RAM), a flash memory, or the like, or a hard disk, an optical disk, or the like. In the present embodiment, the storage 320 is configured to store therein the volume data received from the X-ray diagnosis apparatus 100 or an apparatus in another modality as well as the information about the imaged range to be used during the puncture procedure.

The input interface 330 is connected to the processing circuitry 350 and is configured to convert an input operation received from the operator into an electrical signal and to output the electrical signal to the processing circuitry 350. For example, the input interface 330 is realized by using a trackball, a switch button, a mouse, a keyboard, a touchpad used for performing an input operation by touching the operation surface thereof, a touch screen in which a display screen and a touch pad are integrated together, a contactless input circuit using an optical sensor, as well as an audio input circuit, a foot switch used for causing the radiation of X-rays, and/or the like.

The display 340 is connected to the processing circuitry 350 and is configured to display various types of information and various types of image data output from the processing circuitry 350. For example, the display 340 is realized by using a liquid crystal monitor, a Cathode Ray Tube (CRT) monitor, a touch panel, or the like.

The processing circuitry 350 is configured to control constituent elements of the medical information processing apparatus 300, in accordance with the input operation received from the operator via the input interface 330. For example, the processing circuitry 350 is realized with a processor. In the second embodiment, the processing circuitry 350 is configured to store CT image data output from the communication interface 310 into the storage 320. Further, the processing circuitry 350 is configured to reads CT image data from the storage 320 and to cause the display 340 to display the read CT image data.

Furthermore, as illustrated in FIG. 14, the processing circuitry 350 executes a controlling function 351, an obtaining function 352, an extracting function 353, and a presenting function 354. The controlling function 351 is configured to control the entirety of the medical information processing apparatus 300. Further, the controlling function 351 performs the same processes as those performed by the controlling function 211 described above. The obtaining function 352 performs the same processes as those performed by the obtaining function 212 described above. The extracting function 353 performs the same processes as those performed by the extracting function 213 described above. The presenting function 354 performs the same processes as those performed by the presenting function 214 described above.

In the embodiments described above, the examples are explained in which the single processing circuit (the processing circuitry 21 or the processing circuitry 350) realizes the processing functions; however, possible embodiments are not limited to these examples. For instance, the processing circuitry 21 and the processing circuitry 350 may each be structured by combining together a plurality of independent processors, so that the processing functions are realized as a result of the processors executing the programs. Further, any of the processing functions included in the processing circuitry 21 and the processing circuitry 350 may be realized as being distributed to a plurality of processing circuits or being integrated into a single processing circuit, as appropriate.

The term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The one or more processors realize the functions by reading and executing the programs saved in the storage 25 or the storage 320. In this situation, instead of saving the programs in the storage 25 or the storage 320, it is also acceptable to directly incorporate the programs in the circuits of the processors. In that situation, the processors realize the functions thereof by reading and executing the programs incorporated in the circuits thereof. Further, the processors in the present embodiments do not each necessarily have to be structured as a single circuit. It is also acceptable to structure one processor by combining together a plurality of independent circuits so as to realize the functions thereof.

In this situation, the programs executed by the processors are provided as being incorporated, in advance, into a Read-Only Memory (ROM), a storage unit, or the like. Alternatively, the programs may be provided as being stored on a computer-readable storage medium such as a Compact Disk Read-Only Memory (CD-ROM), a flexible disk (FD), a Compact Disk Recordable (CD-R), a Digital Versatile Disk (DVD), or the like, in a file in such a format that is either installable or executable for the devices. Further, the programs may be stored in a computer connected to a network such as the Internet, so as to be provided or distributed as being downloaded via the network. For example, each of the programs is structured with a module including the functional units explained above. In actual hardware, as a result of a CPU reading and executing the programs from a storage medium such as a ROM, the modules are loaded into a main storage device so as to be generated in the main storage device.

Further, the constituent elements of the apparatuses and the devices illustrated in the drawings for the embodiments above are based on functional concepts. Thus, it is not necessary to physically configure the constituent elements as indicated in the drawings. In other words, the specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Furthermore, all or an arbitrary part of the processing functions performed by the apparatuses and the devices may be realized by a CPU and a program that is analyzed and executed by the CPU or may be realized as hardware using wired logic.

As explained above, according to at least one aspect of the embodiments described above, it is possible to easily establish the setting for the puncture procedure.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis apparatus comprising:
processing circuitry configured to
obtain volume data acquired by imaging a subject by using a first image taking apparatus;
set one or more insertion paths to be used when a device is inserted into the subject, on a basis of the volume data;
obtain an imaged range that is related to the subject and is to be used by a second image taking apparatus when the device is inserted into the subject;
evaluate the one or more insertion paths by comparing the set insertion paths with the imaged range; and
present a result of the evaluation.

2. The medical image diagnosis apparatus according to claim 1, wherein
the second image taking apparatus is an X-ray diagnosis apparatus having an X-ray tube provided on one end of an arm and having an X-ray detector provided on another end of the arm, and
the processing circuitry is configured to obtain the imaged range on a basis of at least a movable range of the arm.

3. The medical image diagnosis apparatus according to claim 1, wherein
the processing circuitry is configured to
set two or more of the insertion paths,
extract such an insertion path that is included in the imaged range, from among the two or more insertion paths, and
present the insertion path included in the imaged range as the result of the evaluation.

4. The medical image diagnosis apparatus according to claim 1, wherein the processing circuitry is configured to extract such an insertion path that is positioned outside the imaged range, from the one or more insertion paths extracted on a basis of anatomical position information.

5. The medical image diagnosis apparatus according to claim 1, wherein
the second image taking apparatus is an X-ray diagnosis apparatus including an arm,
the processing circuitry is configured to
obtain information about a movable range and a reference range of the arm, and
extract, on a basis of the information about the movable range and the interference range of the arm, such an insertion path that is positioned outside the imaged range, from the one or more insertion paths to a target site.

6. The medical image diagnosis apparatus according to claim 1, wherein
the second image taking apparatus is an X-ray CT apparatus including a gantry that is tiltable at a predetermined tilting angle with respect to the subject,
the processing circuitry is configured to
obtain information about the tilting angle of the gantry, and
extract, on a basis of the information about the tilting angle of the gantry, such an insertion path that is positioned outside the imaged range, from the one or more insertion paths to a target site.

7. The medical image diagnosis apparatus according to claim 1, wherein
the second image taking apparatus is an ultrasound diagnosis apparatus including an ultrasound probe configured to transmit and receive an ultrasound wave to and from the subject,
the processing circuitry is configured to
obtain information about a transmitting and receiving state of the ultrasound wave to and from the target site, and
extract, on a basis of the information about the transmitting and receiving state of the ultrasound wave to and from the target site, such an insertion path that is positioned outside the imaged range, from the one or more insertion paths to a target site.

8. The medical image diagnosis apparatus according to claim 5, wherein
the processing circuitry is configured to
obtain information about a position, during a puncture procedure, of a practitioner who performs the puncture procedure on the subject, and
extract, by using the information about the position of the practitioner during the puncture procedure in addition to the information about the imaged range, such an insertion path that is positioned outside the imaged range, from the one or more insertion paths to the target site.

9. The medical image diagnosis apparatus according to claim 1, wherein the processing circuitry is configured to present, in an identifiable manner, the one or more insertion paths to a target site and such an insertion path that is positioned outside the imaged range.

10. A medical information processing apparatus comprising:
processing circuitry configured to
obtain information about an imaged range to be used during a puncture procedure by an image taking apparatus that takes a medical image to be referenced during the puncture procedure;
extract, on a basis of the information about the imaged range, such an insertion path that is positioned outside the imaged range, from one or more insertion paths to a target site subject to the puncture procedure; and
present at least one insertion path obtained by excluding the insertion path positioned outside the imaged range, from among the one or more insertion paths to the target site.

11. A medical information processing method comprising:
obtaining information about an imaged range to be used during a puncture procedure by an image taking apparatus that takes a medical image to be referenced during the puncture procedure;
extracting, on a basis of the information about the imaged range, such an insertion path that is positioned outside the imaged range, from one or more insertion paths to a target site subject to the puncture procedure; and
presenting at least one insertion path obtained by excluding the insertion path positioned outside the imaged range, from among the one or more insertion paths to the target site.
